(19) United States
(12) Patent Application Publication
(10) Pub. No.: US 2007/0275084 A1
(43) Pub. Date: Nov. 29, 2007

(54) Compositions And Methods For The Treatment Of Skin Damage

(76) Inventor: Geoffrey Walter Grigg, Lane Cove (AU)

Correspondence Address: CHRISTIE, PARKER & HALE, LLP, PO BOX 7068, PASADENA, CA, 91109-7068, US

(21) Appl. No.: 10/580,389

(22) PCT Filed: Nov. 22, 2004
(86) PCT No.: PCT/AU04/01627

§ 371 Date: Apr. 25, 2007

(30) Foreign Application Priority Data
Nov. 21, 2003 (AU) 2003906419

(51) Int. Cl. A61K 35/36; A61P 17/16

(52) U.S. Cl. 424/535

(57) ABSTRACT

The present invention provides methods and compositions for use in cosmetic and dermatological formulations. In various embodiments of the present invention, compositions comprising beta-alanyl-L-histidine (carnosine) or derivatives or analogs thereof and basic milk factor (BMF) for prophylaxis and treatment of aged or photodamaged, or damaged, as in wounded or disrupted, skin.

### FIELD OF THE INVENITION

The present inventicn relates to a combination of agents for use in cosmetic and dermatological formulations. The invention particularly relates to the combination of a) beta-alanyl-L-histidine (carnosine) or derivatives or analogs thereof and b) basic milk factor (BMF) for prophylaxis and treatment of aged or photodamaged, or damaged as in wounded or disrupted, skin.

### BACKGROUND OF THE INVENTION

The steady deterioration of the appearance and function of skin with age can be attributed to a combination of genetically determined ageing and the cumulative damage to the skin caused by various environmental factors. A distinction can be drawn between intrinsic ageing (or normal biological ageing), and accelerated or premature ageing as a result of damage induced by environmental factors like photodamage caused by the ultraviolet part of solar radiation on the skin.

Premature ageing is often associated with skin damage caused by environmental factors such as the ultra-violet irradiation of the skin that results from sun exposure, or by exposure to other environmental pollutants or toxins. Damage to the skin by environmental factors serves to aggravate the effects of normal biological ageing, producing more detrimental effects on the function and appearance of the skin. The reduction in the youthful appearance of the skin can also be attributed to the functional and structural deterioration of skin as a result of normal biological ageing, exclusive of environmental factors. Such deterioration can manifest visibly as localised furrows (wrinkles) in the epidermis, a loss of elasticity of the skin leading to sagging, hyperpigmentation, and changes to skin thickness.

Human skin, like the skin of all mammals, contains substantial amounts of fibrous proteins of which the most important are the collagens.

The functions of this latter class of protein are several:

One function is to strengthen skin structure to protect it from damage by environmental factors or from intrusive insult by metabolites or metabolic side products such as reactive oxygen species. Another function relates to the ability of collagens to build water molecules to maintain an appropriate water balance in the skin.

With time, or by exposure to various extraneous chemicals, the skin's ability to manufacture new collagen declines. Moreover, the amount of collagen in the skin declines as a person ages. A seventy year old individual has only about 50% of that of a 20 year old.

During the aging process other changes occur in the skin as a result of the production of reactive oxygen species, non-enzymatic glycation agents and the other metabolites. These substances modify the structure of skin proteins including collagens. These become cross-linked so that in the aged skin the fibrous proteins, instead of lying free in the skin matrix are tied together in a matted form resulting in a loss of skin elasticity and structure.

So aged skin is thinner, more fragile, less elastic, wrinkled and less plump to feel then juvenile skin.

Numerous agents have been proposed to prevent and treat intrinsic and environmental damage to skin. These include alpha-hydroxy acids (AHA), retinoids (vitamin A and its derivatives such as retinoic acid), copper-peptide complexes, and vitamin C. Also, it has already been proposed to employ BMF and agents from milk for the treatment or prevention of photodamage to the skin. Examples for the use of BMF and agents from milk are to be found in specifications PCT/US00/04427 and PCT/AU01/00854 and relate to their use to stimulate collagen synthesis and inhibit the production and/or activity of proteinase enzymes such as matrixmetalloproteinases (MPP) and serine proteinases. The use of agents from milk as skin beautifiers has also been described in Japanese specification 06-293679. In addition, it has been proposed to employ carnosine or analogs or derivatives thereof for the treatment or prevention of photodamage to the skin. Examples for the use of carnosine can be found in specification PCT/EP94/00760 where it is proposed to act as an antioxidant or an agent that can trap free radicals and prevent photoinduced oxidative damage in skin. A further example for the use of carnosine was reported by McFarland and Halliday (Exp Cel Res 212, 167-175 1994) where it has been found to retard the senescence of human flbroblasts and thereby function to restrict the structural deterioration of skin induced by ageing and sun exposure. Carnosine has since been incorporated into cosmetic lotions.

The present inventors have now surprisingly found a mixture of BMF and carnosine enhances the production of collagen by human skin fibroblasts. It will be recognized however by those skilled in the art this finding provides that this combination of active agents may be used as improved cosmetic and dermatological treatments for aged, photodamaged or damaged (wounded) skin.

### SUMMARY OF THE INVENTION

In a first aspect of the present invention there is provided a composition for the stimulation of collagen synthesis by human skin fibroblasts, said composition comprising a mixture of BMF and carnosine.

In a second aspect of the present invention there is provided a composition for the treatment of aged, photodamaged and damaged skin, said composition comprising a mixture of BMF and carnosine.

In another aspect of the present invention there is provided a method for treating aged, photodamaged and damaged skin, said method comprising applying to the skin of a human in need thereof an effective amount of a composition comprising a mixture of BMF and carnosine.

In a further aspect of the present invention there is provided a method for improving the aged appearance of skin, said method including applying to the skin of a human in need thereof an effective amount of a composition comprising a mixture of BMF and carnosine.

In yet a further aspect the present invention provides the use of a composition comprising a mixture of BMF and carnosine in the preparation of a composition for treating aged, photodamaged and damaged skin.

In still a further aspect the present invention provides the use of a composition comprising a mixture of BMF and carnosine in the preparation of a composition for improving the aged appearance of skin.

By enhancing the production of collagen by skin fibroblasts, a mixture of BMF and carnosine improve the effects each individual agent has on aged, photodamaged and damaged skin and may therefore be used to reverse, or at least partially reverses the effects of ageing or skin damage brought about by exposure to environmental factors such as sun exposure and other means such as wounding or skin disruption. This results in the skin exhibiting a more enhanced cosmesis as well as improved structure and function and skin repair, such as in healing and the like.

### FIGURES

FIG. 1 shows a histogram demonstrating the increase in collagen production in human skin fibroblasts incubated with a mixture of BMF and carnosine. After starving for up to 6 hours, cells were exposed to a mixture of BMF and carnosine or the appropriate controls for 48 hours. Tritiated proline (L-[5-³H] proline) was included in the culture medium and at the end of the experiments, the amount of ³H proline incorporated into collagenous protein was determined as an index of collagen synthesis. A subset of cultures were treated with collagenous for the final 6-8 hours of the culture to determine the collagen-specific nature of incorporated radioactivity.

### DETAILED DESCRIPTION OF THE INVENTION

In a first aspect the present invention provides compositions for the treatment of aged, photodamaged or damaged skin, said compositions comprising basic milk factors (BMF) and carnosine.

The term “BMF” as used herein means a mixture of growth factors concentrated from milk, the growth factors having approximately neutral to basic isoelectric points.

In a preferred embodiment, the BMF is BMF-1 and/or BMF-2 or mixtures thereof.

In a further preferred embodiment the BMF to be used for treating the skin is isolated from mammalian milk.

More preferably the BMF is isolated from cheese whey, colostral whey, skim milk or acid whey.

Even more preferably the BMF is isolated from bovine cheese whey.

Most preferably method for obtaining BMF is described in Australian Patent Number 645589 or Australian Patent 702002 the contents of which are incorporated herein.

Preferably, the BMF includes growth stimulating factors selected from the group comprising insulin-like growth factor I (IGF-I), insulin-like growth factor II (IGF-II), platelet derived growth factor (PDGF), fibroblast growth factor (FGF) and Transforming Growth Factor Beta (TGFβ).

Preferably, the growth factors present in the composition are deficient in EGF.

Preferably the basic milk factors are prepared by subjecting a milk product to cationic exchange chromatography, for example, a cationic exchange resin suitable for adsorbing a plurality of cell growth stimulating factors. The cationic exchange resin may be suitable for adsorbing basic proteins such that the more basic components of the milk product are adsorbed thereon. A suitable cationic exchange resin used in accordance to the invention includes an agarose-based cationic exchange resin. A suitable buffer solution of a sufficiently high ionic strength (e.g. a molarity above 0.2M) may be used to elute proteins from the cationic exchange resin. The eluate may be filtered to remove salt or any other low molecular weight contaminants.

In a preferred embodiment the BMF is capable of stimulating proliferation of rat L6 myoblasts.

Carnosine includes beta-alanyl-L-histidine (carnosine) or derivatives or analogs thereof.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. All references, including any patents or patent applications, cited in this specification are hereby incorporated by reference. It will be clearly understood that although a number of prior art publications are referred to herein, this reference does not constitute an admission that any of these documents forms part of the common general knowledge in the art, in Australia or in any other country.

The term “damaged” includes any resultant adverse effect on the skin by way of normal biological ageing, or as a rebut of exposure to environmental factors, or a combination of both. Adverse effects on the skin may manifest visibly as wrinkles, loss of elasticity, sagging, hyperpigmentation, dryness, and changes to skin thickness, and other undesirable dhanges. Also included are adverse effects that are not apparent to the eye. For example deleterious metabolic changes in the skin cells, and changes to skin vascularisation. Also included is wounded skin, thereby including lacerations, penetrations, ulcers and burns and the like. Also included is chaffed, cracked and disrupted skin and the like.

Preferably the damaged skin is the result of normal biological ageing, an environmental factor, a dermatological disorder, medical treatment, surgical treatment or a medical condition, either alone or in combination. The normal biological ageing may be predetermined by genetic factors, whereas an environmental factor may include poor food hygiene, exposure to a toxin, exposure to a pollutant, sun exposure, ionizing radiation, X-rays, UV-rays, tobacco use, alcohol or stress.

The dermatological disorder may arise from acneic conditions, acne vulgaris, acne rusacea, actinic keratoses, actinodernmatoses, angiomas, argyia, chloasma, Darier's disease, dyschromias, lentigines, melasma, nevi, radiodermatitis, rhinophyma, rhytes and rhytides, sebaceous adenomas, sebaceous cysts, seborrheic keratoses, superficial basal cell carcinoma, telangiectasis and/or trichoepitheliomas.

Damaged skin may also result from the administration of medications for the management of medical conditions or surgical treatments. Preferably the medical treatment is topical glucocorticold treatment or hemodialysis treatment. Preferably, the surgical treatment is liposuction, subscision, electrodesiccation and laser therapy. While surgical in nature, such procedures leave the surface of the skin substantially intact, though the dermal structures underneath the intact skin may still be damaged and require treatment.

Damaged skin may also result from a medical condition including congenital ectodermal dysplasia, diabetes, HIV infection, an infection associated with AIDS, a nutritional deficiency, renal disease, menopause, recessive dystrophic epidermolysis bullosa, Ehlers Danlos syndrome, generalised cutaneous atrophy, localised cutaneous atrophy, scarring alopaecia, pyoderma gangrenosum, or a hormonal alteration, either alone or in combination.

As well as “damaged”, the skin may also be “intact” as used herein and refers to skin which has maintained structural and functional integrity. Examples of intact skin include skin showing (or having the potential to show) signs of normal biological ageing, or skin damaged (or having the potential to be damaged) by environmental exposure. Also included is skin which has been subjected to medical or surgical treatment, where the skin is left substantially intact.

In another aspect, the present invention provides pharmaceutical compositions including BMF and carnosine.

The concentration of BMF in pharmaceutical compositions may be from 1 μg/g up to any higher economic concentration and the concentration of carnosine may be from 1 nM up to any higher economic concentration.

Preferably the concentration of BMF in the composition is from about 0.01 mg/g to about 200 mg/g, preferably about 0.01 to about 1.0% (w/w) most preferably about 0.02 to about 0.2% (w/w).

Preferably the concentration of carnosine from about 1 mM to about 100 mM, preferably about 0.1 to about 2.0% (w/v) more preferably about 1.0% (w/v).

More preferably, the compositions further comprises a pharmaceutically acceptable carrier.

The pharmaceutical compositions may be administered in a therapeutically or prophylactically effective amount for treating or preventing ageing, photodamage or damage to the skin.

The term “a therapeutically or prophylactically effective amount” as used herein means that amount necessary to at least partially attain the desired effect, or to delay the onset of, inhibit the progression of, or halt altogether, the onset or progression of ageing photodamage or damage to skin. Such amounts may depend, of course, on the particular condition being treated, the severity of the condition and individual parameter including age, physical condition, size, weight and other concurrent treatments. These factors are well known to those of ordinary skill in the art and can be addressed with no more than routine experimentation. It is generally preferred that a minimum effective dose be determined according to sound medical or therapeutic judgement. It wilt be understood by those of ordinary skill in the art, however, that a higher dose may be administered for medical or other reasons.

In a further aspect, the present invention provides cosmetic compositions containing BMF and carnosine.

Preferably, the concentration of BMF in cosmetic compositions is from 1 μg/g to any higher economic concentration and carnosine 1 nM to any higher economic concentration.

Most preferably, the cosmetic composition further comprises a cosmetically acceptable carrier.

Cosmetic compositions may be administered in a cosmetically effective amount. The term “a cosmetically effective amount” as used herein means that amount necessary to at least partially attain the desired effect, or to delay the onset of, or inhibit the progression of the appearance of aged, photodamaged or damaged skin. Such amounts may depend, of course, on the particular condition being treated, the severity of the condition and individual parameters, including age, physical condition, size, weight and other concurrent treatments. These factors are well known to those of ordinary skill in the art, and may be addressed with no more than routine experimentation. It is generally preferred that a minimum effective dose be determined according to cosmetic judgement.

Methods and carriers for the preparation of pharmaceutical and cosmetic compositions are well known in the art, as set out in textbooks such as Remington's Pharmaceutical Siences, 18^{th} Edition, Mack Publishing Company, Easton, Pa., USA, the contents of which is incorporated herein.

The preparations contemplated by the present invention include any formulations suitable for the cutaneous application of BMF and carnosine. Suitable pharmaceutically acceptable carriers and/or diluents are known to those skilled in the art and include conventional solvents, dispersion media, fillers, aqueous solutions, sunscreens, antibacterial and antifungal agents, absorption-promoting agents, and the like.

Cosmetically acceptable carriers may further include cosmetically acceptable liquids, creams, oils, lotions, ointments, gels, roll-on liquids, skin patches, sprays, glass bead dressings, and synthetic polymer dressings impregnated with BMF and carnosinic, solids, such as conventional cosmetic night creams, foundation creams, suntan lotions, hand lotions, make-up, make-up bases, masks and the like. Except insofar as any conventional medium or agent is incompatible with the active ingredient use thereof in the cosmetic compositions of the present invention is contemplated.

Supplementary active ingredients can also be incorporated into both pharmaceutical and cosmetic compositions, such as additional growth factors, Vitamin A, C and E, dimethylsulfoxide, retinoic acid, copper-peptide complexes, alpha-keto acids, lanolin, vaseline, aloe vera, methyl or propyl paraben, pigments and the like.

In a further aspect of the present invention there is provided a method for treating aged, photodamaged or damaged skin, said method comprising applying to the skin of a mammal in need thereof, an effective amount of BMF and carnosine.

In a preferred embodiment, the treatment reduces the aged, photodamaged, or damaged appearance of skin.

The present invention also provides a method for the prevention of damage to skin, said method comprising applying to a mammal in need thereof an effective amount of at least one basic milk growth factor and carnosine.

In a preferred embodiment the treatment at least partially inhibits further ageing in the appearance of skin.

In all cases the skin to be protected or treated may be damaged or potentially damaged by environmental factors, medical treatment, wounding or resulting from surgery. The damage may result from normal biological ageing of the skin.

Preferably, the skin to be treated has the potential to be damaged or is damaged by exposure to sunlight.

Without being restricted by theory, it is expected BMF and carnosine can permeate through the outer layers of the skin and exert their biological effects on competent cells leading to metabolic changes in those cells. These changes lead to an improvement in the appearance, and/or structure, and/or function, and/or healing of the skin.

The pharmaceutical compositions include sufficient BMF and carnosine so that it may be applied to the skin at a rate sufficient for the delivery of a therapeutically, prophylactically effetive amount of BMF and carnosine.

The frequency of applying the pharmaceutical composition may be sufficient for maintaining the delivery of a therapeutically or prophylactically effective amount of BMF and carnosine to the skin.

More preferably the pharmaceutical composition is administered topically at a rate of 0.1 mg/cm of skin to 2 g/cm of skin.

Preferably, the composition is applied at a frequency necessary to at least partly repair or prevent further damage to aged, photodamaged or damaged skin.

The cosmetic compositions include sufficient BMF and carnosine so that it may be applied to the skin at a rate sufficient for the delivery of a cosmetically effective amount of BMF and carnosine.

Cosmetic compositions may be applied to the skin at a rate sufficient for the delivery of a cosmetically effective amount of BMF and carnosine.

The frequency of applying cosmetic compositions may be sufficient for maintaining the delivery of a cosmetically effective amount of BMF and carnosine to the skin.

Preferably, the composition is applied at a frequency necessary to at least partly reduce or prevent the aged, photodamaged or damaged appearance of skin.

For therapeutic, prophylactic or cosmetic use the composition may be spread or rubbed onto the skin, or left coated on the skin.

In a further aspect of the present invention there is provided a method for improving the aged appearance of skin, said method including applying to the skin of a human in need thereof an effective amount of a composition comprising a mixture of BMF and carnosine.

Preferably, improving the function of the skin includes enhancing the youthful appearance of skin, including the regeneration and/or preservation of existing epithelial, epidermal and dermal tissue, the enhancement of skin flexibility, firmness, smoothness, suppleness and/or elasticity, reducing wrinkles, reducing blemishes, or a combination of any of these.

In another aspect the present invention provides a kit for cosmetic treatment of the aged appearance of skin, comprising a composition as described herein; and instructions for use of the composition in a method as described herein.

The terms “comprise”, “comprises” and “comprising” as used throughout the specification are intended to refer to the inclusion of the stated component or feature or group of components with or without the inclusion of a further component or feature or group of components or features.

The present invention will now be fully described with reference to the following examples. It should be understood however that the description following is illustrative only and should not be taken in any way as a restriction on the generality of the invention described above.

### EXAMPLES

### Example 1

### Production of Basic Milk Factors (BMF) Suitable for the Cosmetic and Therapeutic Treatment of Age, Photodamaged and Damaged Skin

BMF was prepared as in Australian Patent Number 645589. The process involves the microfiltration of pasteurised whey to remove solids, adsorption of growth-promoting material to a column of S-Sepharose Fast Flow S™ cation exchange resin (Pharmacia) that had been equilibrated with 50 mM sodium citrate buffer to remove unabsorbed material, elution of BMF with 0.4M NaCl added to 10 mM sodium citrate pH 6.5, diafltration against water, concentration and if necessary, freeze drying.

### Example 2

### Formulations Suitable for Applying Basic Milk Factors and Carnosine to Skin

All units for ingredients of the compositions are measured in “parts”. The formulations are prepared in a manner well known to those skilled in the art, in particular by mixing the constituents if appropriate at elevated temperatures although care should be taken not to elevate the temperature of solutions containing BMF or carnosine above 60° C. The oily and aqueous phases are prepared separately and mixed or emulsified as necessary.

(i) Cetomacrogol Cream

| Basic milk factor/carnosine product | qs |
|---|---|
| Cetomacrogol emulsifying wax | 15 |
| Liquid paraffin (by weight) | 10 |
| Chlorocresol Propylene glycol | 5 |
| Distilled water to | 100 |

(ii) Aqueous Cream APF

| | |
|---|---|
| Basic milk factor/carnosine product | qs |
| Emulsifying ointment | 30 |
| Glycerol | 5 |
| Phenoxyethanol | 1 |
| Distilled water to | 100 |

(iii) Buffered Cream UPC 73

| | |
|---|---|
| Basic milk factor/carnosine product | qs |
| Citric acid | 5 |
| Sodium phosphate | 25 |
| Chlorocresol | 1 |
| Emulsifying ointment | 30 |
| Distilled water to | 100 |

(iv) Emulsifying Ointment APF

| | |
|---|---|
| Basic milk factor/carnosine product | qs |
| Emulsifying wax | 30 |
| White soft paraffin | 50 |
| Liquid paraffin (by weight) | 20 |

(v) Peptide Ointment (as in Neomycin and Bacitracin Ointment BPC 73)

| | |
|---|---|
| Basic milk factor/carnosine product | qs |
| Liquid paraffin | 10 |
| White soft paraffin to | 100 |

(vi) Gel (as used in Lignocaine and Chlorhexidine Gel APF)

| | |
|---|---|
| Basic milk factor/carnosine product | qs |
| Tragacanth | 2.5 |
| Glycerol | 25 |
| Distilled water to | 100 |

(vii) Spray (as used in Adrenaline and Atropine Spray BPC 73)

| | |
|---|---|
| Basic milk factor/carnosine product | qs |
| Sodium metabisulphite | 1 |
| Chlorbutol | 5 |
| Propylene glycol | 50 |
| Distilled water to | 1000 |

(viii) Cetomacrogol Lotion APF

| | |
|---|---|
| Basic milk factor/carnosine product | qs |
| Cetomacrogol emulsifying wax | 3 |
| Liquid paraffin | 10 |
| Paraffin oil, DAB 9 | 14 |
| Propylene glycol | 3.8 |
| Magnesium sulphate | 0.7 |
| Distilled water to | 100 |

(xii) O/W Emulsion

| | |
|---|---|
| Basic milk factor/carnosine product | qs |
| PEG 100 stearate (Arlacel 185) | 5 |
| Cetearyl alcohol (Lanette O) | 3 |
| Mineral oil, DAB 9 | 25 |
| Paraben mixture | as required |
| Distilled water to | 100 |

(xiii) O/W Emulsion

| | |
|---|---|
| Basic milk factor/carnosine product | qs |
| Polysorbate 60 (Tween 60) | 3 |
| Sorbitan stearate (Arlacel 60) | 2 |
| Cetearyl alcohol (Lanette O) | 3 |
| Mineral oil, DAB 9 | 25 |
| Paraben mixture | as required |
| Distilled water to | 100 |

(xiv) Cationic Emulsion

| | |
|---|---|
| Basic milk factor/carnosine product | qs |
| Distearyldimethylammonium chloride | 5 |
| Vaseline, DAB 9 | 5 |
| Isopropyl palmitate | 2 |
| Getyl alcohol | 1 |
| Silicone oil | 0.1 |
| Propylparaben | 0.1 |
| Methylparaben | 0.1 |
| Glycerol | 10 |
| Chlorhexidine gluconate solution | 0.1 |
| Distilled water to | 100 |

(ix) W/O Cream

| | |
|---|---|
| Basic milk factor/carnosine product | qs |
| Glycerol sorbitan fatty acid ester (Arlacel 481) | 6 |
| Microcrystalline wax (Lumacera M) | 1 |
| Neutral oil | 3 |
| Paraffin oil | 19 |
| Magnesium stearate | 1 |
| Propylene glycol | 3.7 |
| Magnesium sulphate | 0.7 |
| Distilled water to | 100 |

(x) W/O Emulsion

| | |
|---|---|
| Basic milk factor/carnosine product | qs |
| Pvlyoxyethylene glycerol sorbitan fatty acid | 3.6 |
| Ester (Arlacel 988) | |
| Polyoxyethylene fatty acid ester (Arlacel 989) | 1.4 |
| Cetearyl alcohol (Lanette O) | 2 |
| Mineral oil, DAB 9 | 25 |
| Paraben mixture | as desired |
| Magnesium sulphate | 0.7 |
| Distilled water to | 100 |

(xi) W/O Lotion

| | |
|---|---|
| Basic milk factor/carnosine product | qs |
| Glycerol sorbitan fatty acid ester (Arlacel 481) | 1.3 |
| Polyoxyethylene fatty acid ester (Arlacel 989) | 3.7 |
| Neutral oil (Myglyol) | 6 |
| Glycerol | 4 |
| Distilled water to | 100 |

(xv) Ionic Emulsion

| | |
|---|---|
| Basic milk factor/carnosine product | qs |
| Sodium cetearyl sulphate (Emulgade F) | 6 |
| Mineral oil, DAB 9 | 25 |
| Paraben mixture | as required |
| Distilled water to | 100 |

(xvi) Ionic O/W Emulsion

| | |
|---|---|
| Basic milk factor/carnosine product | qs |
| Stearic acid | 5 |
| Cetearyl alcohol (Lanette O) | 3 |
| Mineral oil, DAB 9 | 25 |
| Paraben mixture | as required |
| Triethanolamine | 1 |
| Distilled water to | 100 |

(xvii) Aqueous Formulation (Face Lotion)

| | |
|---|---|
| Basic milk factor/carnosine product | qs |
| PEG 40-hyrdrogenated castor oil | 0.811 |
| Dipropylene glycol | 2.534 |
| PEG 8 | 1.521 |
| Na₃EDTA | 0.253 |
| Polymer JR 125 | 0.025 |
| Distilled water to | 100 |

(xviii) Aqueous Composition

| | |
|---|---|
| Basic milk factor/carnosine product | qs |
| Poly-fatty acid ester (Cetiol HE) | 16 |
| PPG 3 myristyl ether (Witconol APM) | 1 |
| Propylene glycol | 3 |
| Glycerol | 40 |
| Distilled water to | 100 |

(xix) Formulation of High Water Content (Very Soft)

| | |
|---|---|
| Basic milk factor/carnosine product | qs |
| Ceteareth (Cremophor A 25) | 0.1 |
| Cetearyl alcohol (Lanette O) | 0.4 |
| Vaseline, DAB 9 | 12.5 |
| Mineral oil, DAB 9 | 11 |
| Ceteareth-6-stearyl alcohol (Cremophore A6) | 6 |
| Distilled water to | 100 |

(xx) Formulation of High Water Content (Soft)

| | |
|---|---|
| Basic milk factor/carnosine product | qs |
| Ceteareth-25 (Cremophor A 25) | 1.5 |
| Cetearyl alcohol (Lanette O) | 8.5 |
| Distilled water to | 100 |

(xxi) Formulation of High Water Content (Soft)

| | |
|---|---|
| Basic milk factor/carnosine product | qs |
| Ceteareth-25 (Cremdphor A 25) | 2 |
| Cetearyl alcohol (Lanette O) | 8 |
| Vaseline, DAB 9 | 10 |
| Mineral oil, DAB 9 | 10 |
| Distilled water to | 100 |

(xxii) Formulation of High Water Content (Medium-Firm)

| | |
|---|---|
| Basic milk factor/carnosine product | qs |
| Ceteareth-25 (Cremophor A 25) | 3 |
| Cetearyl alcohol (Lanette O) | 17 |
| Distilled water to | 100 |

(xxiii) Thinly Liquid Lotion

| | |
|---|---|
| Basic milk factor/carnosine product | qs |
| Ceteareth-25 (Cremophor A 25) | 1 |
| Ceteareth-6-stearyl alcohol | 1 |
| Glycerol mono/distearate (Tegin normal) | 2 |
| Cetyl alcohol | 1 |
| Isopropyl myristate | 1.45 |
| Glycerol | 1 |
| Polyvinylpyrrolidone | 0.5 |
| Distilled water to | 140 |

(xxiv) Viscous Lotion

| | |
|---|---|
| Basic milk factor/carnosine product | qs |
| Ceteareth-25 (Cremophor A 25) | 2 |
| Cetearyl alcohol (Lanette d) | 3 |
| Mineral oil, DAB 9 | 5 |
| Propylene glycol | 3 |
| Polyvinylpyrrolidone | 0.5 |
| Distilled water to | 100 |

### Example 3

### Carnosine Enhances Collagen Synthesis by Human Skin Fibroblast Cells Stimulated with BMF

Human diploid ftbroblasts were obtained from neonatal foreskins collected from the Womens' and Chldrens' Hospital (North Adelaide, Australia). The fibroblast cultures were maintained in Dulbecco's Modified Eagle Medium (DMEM) prepared according to the manufacturer's instructions (GIBCO, Invitrogen Corporation), and supplemented with 100U/ml penicllin (GJBCO, Invitrogen Corporation), 100 μg/ml streptomycin (CIBCO, Invitrogen i Corporation), 10 mM HEPES (Sigma-Aldrich) and 10% Foetal Bovine Serum (FBS; Thermo Trace Ltd). Cultures were kept in a humidified 37° C. incubator with 5% CO₂.

Experiments were performed using fibroblasts of a low passage number (14-24).

Fibroblasts were seeded at a density of 6×10⁴ cells/ml in 24-well tissue culture plates and 2-4 replicate wells per treatment performed per experiment.

The fibroblasts were allowed to adhere overnight and the following day starved of FBS for 4-6 h prior to commencemnent of the experiment BMF (0.2 and 2 mg/ml) and carnosine (20-60 mM) either alone or in combination were added to the control medium (DMEM supplemented with 0.1% FBS) together with L-[5-³H]proline radiolabel (50μCi/ml; Amersham Pharmacia) and incubated for 48 h in a humidified 37° C. incubator with 5% CO₂.

Collagenase Type V (1 mg/ml) from Clostridium histolyticum (Sigma-Aldrich) was added to selected wells for the final 6-8 h of the 48 h culture period. The culture medium (fraction containing soluble collagen) was collected and stored at −70° C.

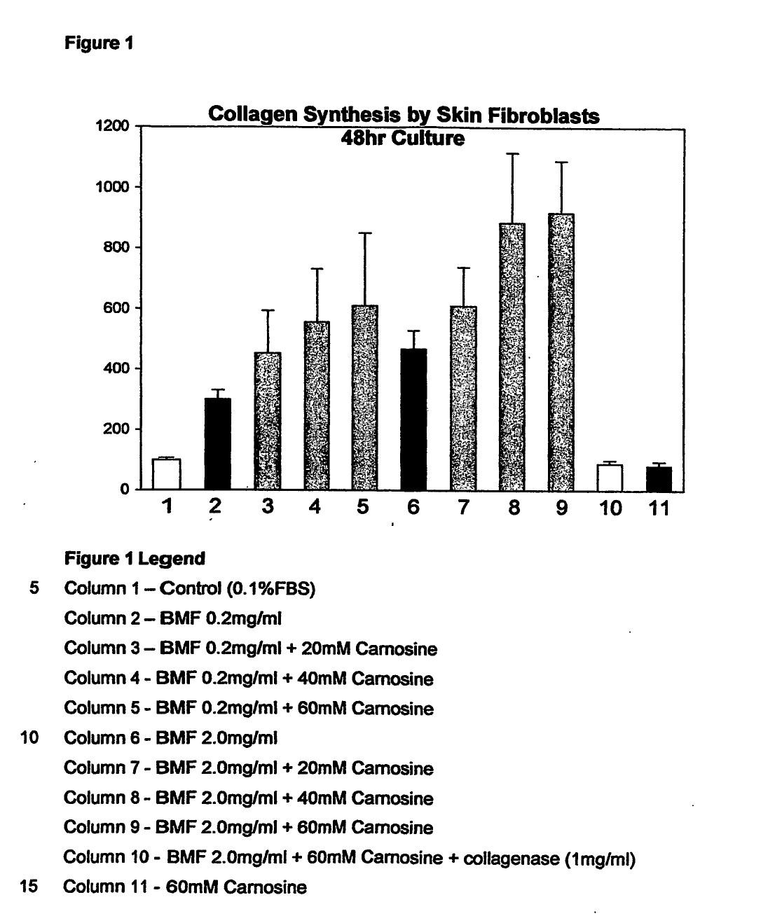

Collagen was purified from the culture medium by successive precipitation with salt solutions at acid and neutral pH. All steps were performed on ice using pre-chilled solutions. All centrifugation steps were carried out at 2000 rpm for 30 minutes at 4° C. 500 μl of 1M glacial acetic acid containing 1 mg/ml pepsin A (Sigma-Aldrich) was added to the culture medium and incubated at 4° C. for approx 16-24 h with gentle agitation. 0.5M glacial acetic acid containing 200 μg/ml add soluble calf skin collagen (Sigma-Aldrich) was then added at 4-times the volume and the samples centrifuged. The supernatants were collected into fresh tubes and the collagen precipitated by addition of 25% NaCl (in 0.5M glacial acetic acid) overnight at 4° C. The samples were centrifuged and the collagen pellet resuspended in 0.15M NaCl (in 0.05M Tris-HCl pH 7.5). Collagen was re-precipitated by the addition of 4.5M NaCl (in 0.05M Tri-HCl pH 7.5) and incubation overnight at 4° C. Samples were centrifuged and the collagen pellet washed with 20% ethanol. The centrifugation step was repeated and the collagen resuspended in 0.5M acetic acid. Samples were diluted in Ultima Gold liquid scintillation fluid (Packard BioScicnce) and mixed thoroughly by repeated inversion. The amount of radioactivity in the samples was determined using a Stcinillation counter (Wailac) as an index of the amount of new collagen synthesised. The radioactivity in each replicate well was normalised as a percentage of the average radioactivity present in the control wells from each experiment. The normalised values of wells from 3-4 experiments were combined (n=8-12) and the mean±scm (standard error of the mean) shown in FIG. 1.

BMF (0.2 and 2.0mg/ml) stimulated collagen production by human skin fibroblasts in a dose dependent manner compared to control (FIG. 1; columns 2 and 6 vs column 1) as previously described in PCT/AU01/00854. When cells were treated with each dose of BMF in combination with carnosine (20-60 mM) a markedly greater amount of collagen was synthesised by the cells (FIG. 1; columns 3-5 vs column 2 and columns 7-9 vs column 6). Carnosine (60 mM) did not stimulate collagen synthesis (column 11) and when collagenase was added at the end of the experiment to wells treated with BMF (2 mg/ml) and carnosine (60 nM), the measured radioactivity was reduced to control levels (column 9 vs 10) indicating that the radio-labelled proline was indeed incorporated into collagenous protein. Thus carnosine enhanced BMF stimulated collagen secretion by human fibroblast cells.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A composition for the stimulation of collagen synthesis by human skin fibroblasts, said composition comprising a mixture of BMF and carnosine.

2. A composition according to claim 1 wherein the concentration of camosine is about 1 mM to about 100 mM.

3. A composition according to claim 1 wherein the composition comprises about 0.1 to about 2.0% (w/v) carnosine.

4. A composition according to claim 3 wherein the composition comprises about 1.0% (w/v) carnosine.

5. A composition according to claim 1 wherein the composition comprises about 0.01 mg/g to about 200 mg/g BMF.

6. A composition according to claim 1 wherein the composition comprises about 0.01 to about 1.0% (w/w) BMF.

7. A composition according to claim 6 wherein the composition comprises about 0.02 to about 0.2% (w/w) BMF.

8. A composition according to claim 1 wherein the BMF includes growth factors selected from the group comprising IGF-1, IGF-2, PDGF, FGF and TGFβ

9. A composition according to claim 1 comprising additional growth factors, Vitamin A, vitamin C , vitamin E, dimethylsulfoxide, retinoic acid, copper-peptide complexes, alpha-keto acids, lanolin, vaseline, aloe vera, methyl or propyl paraben, and pigments alone or in combination.

10. A method for treating aged, photodamaged and damaged skin, said method comprising applying to the skin of a human in need thereof an effective amount of a composition according to claim 1.

11. A method for improving the aged appearance of skin, said method including applying to the skin of a human in need thereof an effective amount of a composition according to claim 1.

12. (canceled)

13. (canceled)
